(19) United States
(12) Patent Application Publication
(10) Pub. No.: US 2009/0005755 A1
(43) Pub. Date: Jan. 1, 2009

(54) GUIDE WIRE CONTROL CATHETER FOR CROSSING OCCLUSIONS AND RELATED METHODS OF USE

(76) Inventors: Peter T. Keith, St. Paul, MN (US); Dennis W. Wahr, Ann Arbor, MI (US); Thomas V. Ressemann, St. Cloud, MN (US); David J. Blaeser, Champlin, MN (US); Timothy B. Petrick, Brocklyn Park, MN (US); Steven S. Hackett, Maple Grove, MN (US)

Correspondence Address: O''Melveny & Myers LLP;IP&T Calendar Department LA-1118, 400 South Hope Street, Los Angeles, CA, 90071-2899, US

(21) Appl. No.: 12/207,391

(22) Filed: Sep. 9, 2008

Related U.S. Application Data
(63) Continuation of application No. 10/301,779, filed on Nov. 22, 2002.

(51) Int. Cl. A61M 25/09; A61M 25/10

(52) U.S. Cl. 604/509; 604/510

(57) ABSTRACT

A wire control catheter for aligning and guiding a guide wire through a lesion in a vessel is provided. The wire control catheter includes a shaft having a guide wire lumen and a control wire lumen. A control wire passes through the control wire lumen and is used in combination with an articulation structure to deflect or curve a distal tip portion of the catheter. The distal catheter shaft may include a centering device for centering the catheter within the vessel. The distal catheter shaft may also include a pre-dilation balloon for dilating the lesion prior to performing angioplasty or other treatment on the lesion. A method of treatment of a blood vessel includes inserting a guide wire into the blood vessel and advancing a control wire over the guide wire until the distal tip of the catheter is near the occlusion in the blood vessel. The tip of the catheter then is deflected via a control wire and an articulation structure. The guide wire is then advanced across the occlusion. The control wire also may be advanced across the occlusion simultaneously with the guide wire or subsequent to the guide wire crossing. Prior to crossing the occlusion, the wire control catheter may be centered using a centering device. Subsequent to crossing the occlusion, the occlusion may be pre-dilated with a pre-dilation balloon of the wire control catheter.

This is a continuation of U.S. application Ser. No. 10/301,779, entitled “Guide Wire Control Catheter for Crossing Occlusions and Related Methods of Use,” filed Nov. 22, 2002, which is expressly incorporated herein by reference in its entirety for all purposes.

### FIELD OF THE INVENTION

The present invention relates to apparatus and methods used to cross lesions in blood vessels, and in more particular embodiments, catheters for controlling a guide wire to cross a chronic total occlusion in a blood vessel.

### BACKGROUND OF THE INVENTION

Chronic Total Occlusions (CTOs) are vascular lesions which are totally occluded and thereby inhibit normal blood flow. Such occlusions can occur anywhere in a patient's vascular system, arteries, and veins, including coronary vessels, as well as carotids, renals, cerebrals, iliacs, femorals, popliteals, and other peripheral arteries.

Typically, a CTO may be occluded for several weeks to several months, or longer. Such blockages can have serious medical consequences, depending upon their location within a patient's vascular system. For example, blockage of the coronary vessels that supply blood to the heart can cause damage to the heart

Since most lesions form episodically over a long period of time, the ischemic tissue distal of the lesion has time to form some collateral circulation. In the case of coronary arteries, these collaterals can form from the proximal artery and connect into the distal artery (“ipsilateral collaterals”) or can form from the other major arterial branches and connect into the distal artery (“contralateral collaterals”). When the lesion finally becomes a total occlusion, the collateral circulation is typically sufficient to keep the distal tissue alive, but ischemic. In cardiac circulation, this ischemic tissue causes angina. Therefore, it is desirable to reestablish flow to the distal tissue.

Various surgical procedures are currently used to reestablish flow through or around the blockage in blood vessels. Such procedures include coronary artery bypass surgery and balloon angioplasty. Balloon angioplasty typically involves inserting a balloon catheter over a guide wire and into the occlusive lesion, expanding the balloon in the lesion, and if necessary, placing a stent in the now expanded lesion to keep it open.

Chronic total occlusions, such as occlusion 10 in vessel 12 shown in FIG. 1A, are more difficult to cross than non-totally occluded lesions because a guide wire, such as guide wire 14, must penetrate the lesion tissue, rather than navigate a pre-existing lumen. Complications may result. For example, as shown in FIG. 1B, the distal end and tip of the guide wire 14 may have insufficient support or rigidity to enter the lesion, causing the end to buckle. Or, guide wire 14 may perforate vessel 12, as shown in FIG. 1C, especially when the distal end and tip of guide wire 14 is not oriented towards occlusion 10. If guide wire 14 has a pre-formed bend 14a at the tip to assist in its initial orientation as it enters the occlusion 10, the internal lesion tissue may cause the guide wire 14 to take an unwanted path within occlusion 10, as shown in FIGS. 1D and 1E. If the guide wire cannot successfully cross the occlusion, subsequent therapeutic devices, such as a balloon angioplasty catheter, cannot be advanced across the occlusion to dilate and treat it.

FIGS. 1F-1H show similar problems when attempting to cross an occlusion 10 at a bifurcation. FIG. 1G shows the distal end and tip of the guide wire 14 having insufficient support or rigidity to enter the lesion, causing the end to bend, and FIG. 1H shows guide wire 14 perforating the vessel at the bifurcation.

For these reasons, the success rate for crossing and treating CTOs is much lower than that for non-totally occluded lesions, particularly for coronary CTOs. Furthermore, even when the total occlusion is successfully crossed with conventional guide wires, it often requires a great deal of time and skill on the part of the physician. Thus, there is a need for an improved system and method of crossing an occlusion.

### SUMMARY OF THE INVENTION

In accordance with the invention, methods and apparatuses for crossing an occlusion are provided.

According to one aspect of the invention, a wire control catheter for controlling advancement of a guide wire through a blood vessel is provided. The wire control catheter comprises a single control wire for articulating a distal tip portion of the catheter, and a shaft having a single control wire lumen for receiving the single control wire.

According to another aspect of the invention, a wire control catheter for controlling advancement of a guide wire through a blood vessel comprises a shaft defining a guide wire lumen and a control wire lumen and having a deflectable distal tip portion, means for deflecting the distal tip portion, and a centering device on a distal portion of the shaft.

According to a further aspect of the invention, a wire control catheter for controlling advancement of a guide wire through a blood vessel comprises a first shaft portion defining a control wire lumen extending between a distal tip of the catheter and a proximal end of the catheter, a second shaft portion defining a guide wire lumen, wherein the guide wire lumen is substantially shorter than the control wire lumen, and a deflectable distal tip portion.

According to yet another aspect of the invention, a system for controlling advancement of a guide wire through a blood vessel is provided. The system comprises a wire control catheter having a guide wire lumen, a control wire lumen, and a control wire within the control wire lumen, and a sliding sheath catheter positionable within the guide wire lumen.

According to another aspect of the invention, a method of treating a blood vessel is provided. The method includes inserting a guide wire into the blood vessel, advancing a control catheter over the guide wire until a distal tip of the catheter is near an occlusion in the blood vessel, deflecting a distal tip of the catheter, and advancing the guide wire across the occlusion.

According to a further aspect of the invention, a wire control catheter for controlling advancement of a guide wire through a blood vessel includes a shaft having a deflectable distal tip, and a pre-dilation balloon connected to a portion of the shaft.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. In the drawings,

FIGS. 1A-1H are cross-section views of occluded vessels showing guide wires attempting to cross the occlusions in those vessels;

FIGS. 2A-2C are cross section views of an occluded vessel showing a guide wire crossing the occlusion through use of a control catheter, according to one embodiment of the present invention;

FIG. 2D is a cross section view of an occluded vessel showing a guide wire and control catheter crossing the occlusion, according to one embodiment of the present invention;

FIG. 2E is a cross section view of an occluded vessel showing a guide wire centered and crossing the occlusion through use of a control catheter, according to another embodiment of the present invention;

FIG. 2F is a cross section view of an occlusion near a bifurcation showing a guide wire crossing the occlusion through use of a control catheter, according to another embodiment of the present invention;

FIGS. 3A-3C are cross section views of an occluded vessel showing centering of a control catheter relative to the occlusion, according to an embodiment of the present invention;

FIG. 4 is a cross section view of an occluded vessel prior to centering of a control catheter relative to the occlusion, according to an embodiment of the present invention;

FIGS. 5A and 5B are cross section views of an occluded vessel showing a guide wire crossing the occlusion through use of a control catheter having a centering element, according to embodiments of the present invention;

FIGS. 6A-6D are cross section views of an occluded vessel showing a guide wire crossing the occlusion through use of a control catheter and a sliding sheath, according to an embodiment of the present invention;

FIG. 7 is a cross section view of the distal end of a control catheter, according to an embodiment of the present invention;

FIGS. 8A and 8B are side and bottom views, respectively, of an articulation structure for use in a control catheter, according to an embodiment of the present invention;

FIGS. 8C and 8D are side and bottom views, respectively, of an alternative articulation structure for use in a control catheter, according to another embodiment of the present invention;

FIG. 8E is a side view of an alternative articulation structure for use in a control catheter, according to yet another embodiment of the present invention;

FIG. 9A is a cross section view of a portion of a control catheter, according to an embodiment of the present invention;

FIG. 9B is a cross section view of the control catheter of FIG. 9A taken along line B-B;

FIG. 9C is a cross section view of a portion of a control catheter, according to another embodiment of the present invention;

FIG. 9D is a cross section of a junction between a distal shaft and an articulation structure of a control catheter, according to an embodiment of the present invention;

FIG. 9E is a junction between a proximal shaft and a distal shaft of a monorail style control catheter, according to one aspect of the present invention;

FIG. 10 is a simplified side view of an over-the-wire style control catheter, with its tip deflected, according to an embodiment of the present invention;

FIGS. 11A and 11B are simplified side views of a monorail style control catheter with its tip undeflected and deflected respectively, according to an embodiment of the present invention;

FIG. 12A is a simplified side view of an over-the-wire style control catheter with an inflatable centering element, according to an embodiment of the present invention;

FIG. 12B is a simplified side view of an over-the-wire style control catheter with an alternative centering element, according to another embodiment of the invention;

FIG. 12C is a simplified side view of a monorail style control catheter with an inflatable centering element, according to an embodiment of the present invention;

FIG. 12D is a simplified side view of a monorail style control catheter with an alternative centering element, according to another embodiment of the present invention;

FIG. 12E is a simplified side view of a monorail style control catheter with a wire centering element, according to an embodiment of the present invention;

FIG. 13A is a simplified side view of an over-the-wire style control catheter having a pre-dilation balloon, according to an embodiment of the invention;

FIG. 13B is a cross section of the proximal shaft of the over-the-wire style control catheter of FIG. 1 3A taken along line B-B;

FIG. 13C is a side view of an over-the-wire style control catheter having a pre-dilation balloon and an inflatable centering element, according to an embodiment of the invention;

FIG. 13D is a cross section of the proximal shaft of the over-the-wire style control catheter of FIG. 13C taken along line D-D;

FIG. 14A is a simplified side view of a monorail style control catheter prior to receiving a sliding sheath, according to an embodiment of the present invention;

FIG. 14B is a simplified side view of a full length style sliding sheath, according to an embodiment of the present invention;

FIG. 14C is a simplified side view of a monorail style sliding sheath, according to an embodiment of the present invention;

FIG. 15 is a simplified side view of the sliding sheath of FIG. 14B assembled with the control catheter of FIG. 14A, according to an embodiment of the present invention;

FIG. 16 is a cross section view of a control catheter having an inflatable centering device proximate its articulation structure, according to an embodiment of the present invention; and

FIG. 17 is a cross section view of a handle structure to be used with a control catheter, according to an embodiment of the present invention.

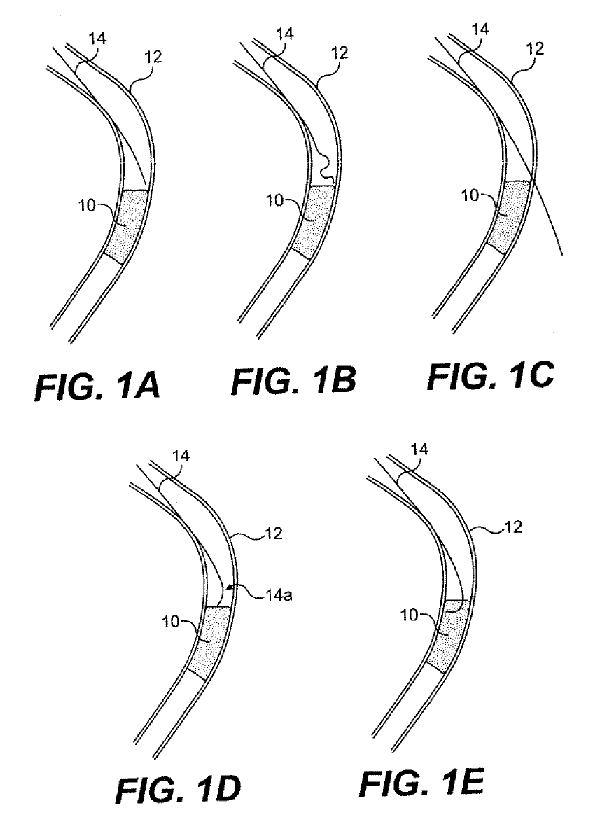

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

According to embodiments of the present invention, systems and methods are provided in which additional support is provided to the flexible end region of a guide wire during advancement of the wire across a lesion in a blood vessel. According to further embodiments, systems and methods are provided in which the direction of advancement of the guide wire tip during crossing of the lesion is controlled These embodiments should improve the success of crossing of the lesion, while minimizing the risk of perforating the blood vessel or crossing into subintimal tissue.

As used herein, an “occlusion,” “blockage,” “stenosis,” or “lesion” refers to both complete and partial blockages of the vessels, stenoses, emboli, thrombi, plaque, debris and any other particulate matter which at least partially occludes the lumen of the blood vessel. Additionally, as used herein, “proximal” refers to the portion of the apparatus closest to the end which remains outside the patient's body, and “distal” refers to the portion closest to the end inserted into the patient's body.

The disclosed methods and systems are particularly suited to be used in diseased blood vessels, including diseased saphenous vein grafts (SVGS), carotid arteries, coronary arteries, renal arteries, cerebrals, iliacs, femorals, popiteals, and other peripheral arteries. However, it is contemplated that the methods and systems can be adapted to be used in other areas, such as other blood vessels.

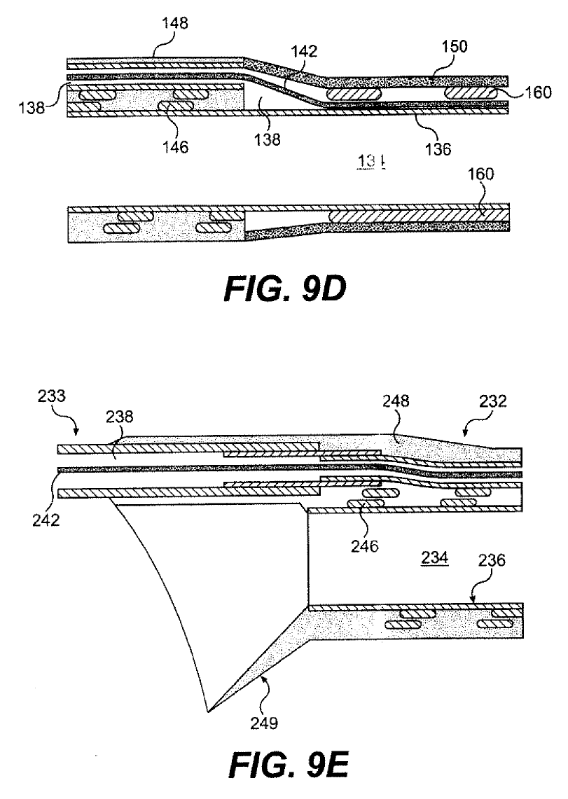

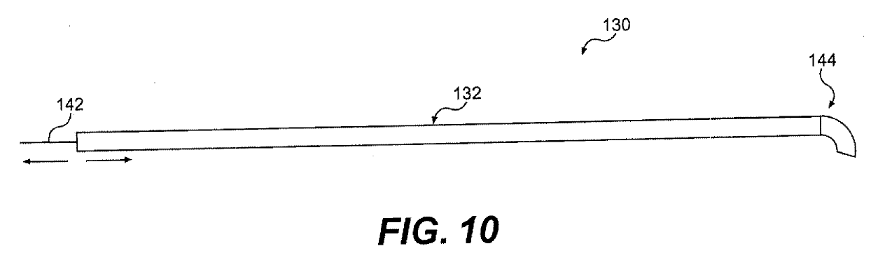

According to one aspect of the present invention, a wire control catheter is provided to guide and support a guide wire through a blockage. As embodied herein and shown in FIG. 10, a preferred embodiment of an over-the-wire (OTW) style catheter 130 is disclosed. OTW catheter 130 includes a full length shaft 132 with a guide wire lumen 134 (see FIGS. 9A and 9B). “Full length” indicates that the guide wire extends within the entire length of the shaft 132 to a proximal end at a handle assembly (not shown in FIG. 10) used to control the catheter 130 and guide wire 114 (not shown in FIG. 10).

As shown in FIGS. 9A and 9B, the guide wire lumen 134 is preferably formed by a lubricious inner liner 136 made of, for example, PTFE, to allow for ease of movement of a guide wire 114 within the lumen 134. Shaft 132 further includes a lumen 138 for a control wire 142. The control wire 142 controls articulation of a directable distal tip section 144 of OTW catheter 130, to be described in more detail below. Control wire lumen 138 also may include a lubricious liner 140. The lubricious liners 136, 140 may be individual tubes that form the guide wire lumen 134 and control wire lumen 138. These individual tubes 136, 140 may be surrounded by a wire braid 146 that imparts torsional stiffness to OTW catheter 130. FIG. 9B shows a braid 146 surrounding only liner 136. The wire braid 146 is preferably metallic, made for example of a metallic ribbon of stainless steel. Preferably, the metallic material is a ribbon having the dimensions of about 0.001 inch by about 0.003 inch to about 0.008 inch. The pick count can be varied along the length of the shaft to further alter the stiffness and torsional stiffness qualities of the shaft.

A polymeric jacket 148 may surround and encapsulate the braid 146, and is preferably made of a thermoplastic such as nylon, Pebax, polyurethane, PEEK (polyether ether ketone), or a thermoset such as silicone or polyimide. Preferably, polymer jacket 148 includes multiple grades of one or more of these polymers to result in a gradual change in stiffness along the length of the catheter, the stiffness changing from relatively stiff at the proximal portion of shaft to more flexible (i.e., relatively less stiff) near the distal end. For example, the distal most portion of the shaft may incorporate an encapsulation of a relatively flexible polymer such as a soft durometer polyurethane, and progress to more rigid polyurethanes or Pebax, progress to Nylon, and then to a polyimide encapsulation. Any number and composition of encapsulation materials are contemplated to tailor the shaft stiffness and torsional stiffness qualities at various positions along the length of the shaft. Polymer jacket 148 may further include a lubricious coating such as a hydrophilic coating. Alternatively, the wire braid 146 may surround both individual tubes 136, 140, that form, respectively, the guide wire lumen 134 and the control wire lumen 138, as shown in FIG. 9C. In this case, polymer jacket 148 may extend through the braid 146 to the liner 136, or may encapsulate only the braid 146.

The diameter of catheter 130 is designed to accommodate a guide wire 114 and a control wire 142. For coronary applications, catheter 130 is preferably sized to accommodate guide wires of about 0.014 inch, but may be dimensioned to work with larger or smaller diameter guide wires. To accommodate a 0.014 inch guide wire, liner 136 is preferably 0.015 inches to 0.017 inches in diameter, and most preferably is about 0.016 inches. An outer diameter of catheter 130 is preferably about 0.020 inches to about 0.060 inches, and most preferably is about 0.022 inches to about 0.040 inches.

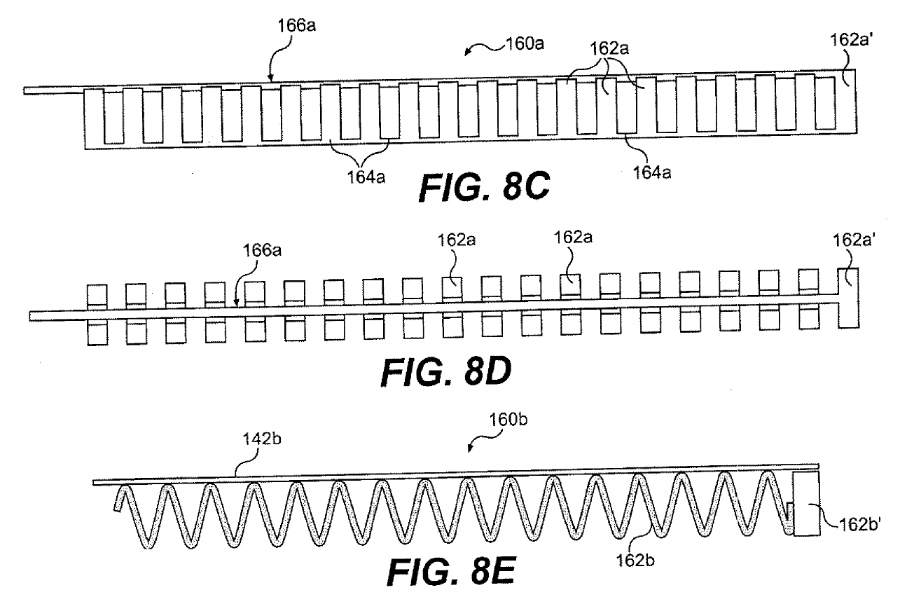

According to one aspect of the present invention, the OTW style catheter 130 includes a variably deflectable tip 144. The deflectable tip 144 is controlled by control wire 142. FIG. 7 shows an embodiment of a deflectable tip 144 of a wire control catheter 130. The deflectable tip 144 includes an outer tube 150, preferably a flexible, thin walled lubricious tube made of, for example, PTFE, ePTFE, HDPE, polyurethane, silicone, or other lubricious polymer. Tube 150 has an inner liner 136 defining the guide wire lumen 134. The liner 136 preferably extends the entire desired length of the guide wire lumen 134 through the shaft 132 of the catheter 130. Near the distal end of liner 136 is a marker 154, which is preferably a short tubing of radiopaque material such as platinum or platinum alloy. The end of the deflectable tip 144 may include a tapered tip portion 156 that may be formed by a backfill of a suitable adhesive, such as polyurethane or epoxy.

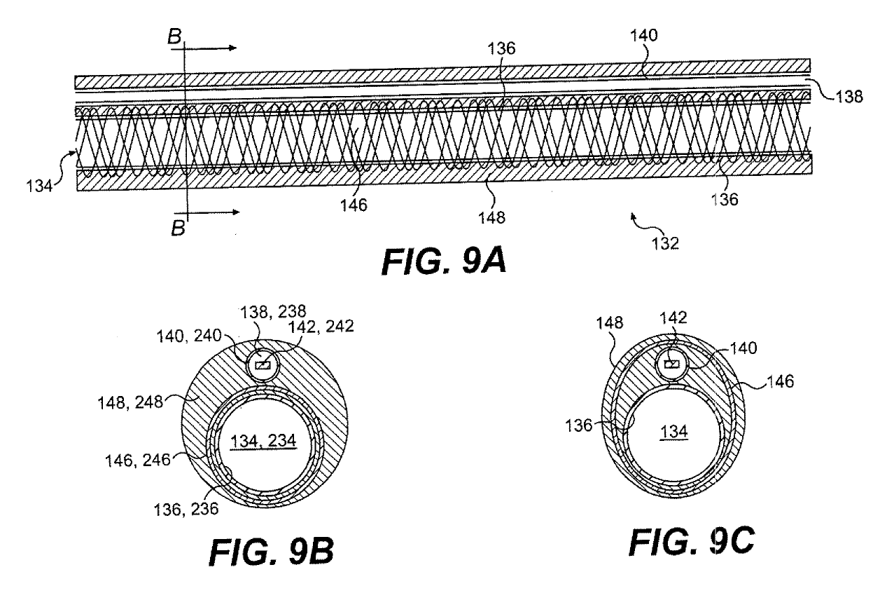

Proximal of marker 154, and surrounding liner 136, is an articulation structure 160. The deflectable tip 144 shown in FIG. 7 includes the articulation structure 160 shown in FIGS. 8A and 8B. As embodied herein and shown in FIGS. 8A and 8B, articulation structure 160 is tubular and incorporates a series of rings 162 connected to a longitudinally extending spine 164. Articulation structure 160 may be fabricated by laser cutting a metallic tube, preferably stainless steel, or by other suitable methods. Articulation structure 160 is configured to bend when the side of the structure opposite that of spine 164 is foreshortened. Rings 162 deflect towards one another on the foreshortened side, while spine 164 prevents such foreshortening on the opposite side. Rings 162 further serve to prevent liner 136 from kinking when the tip is deflected into a curved position.

Articulation structure 160 is activated by longitudinal motion of control wire 142. Control wire 142 preferably passes through articulation structure 160 and is secured to the distal most ring 162′, either directly, or via a direct connection with the abutting tubular marker 154, as is shown in FIG. 7. Control wire 142 extends to the proximal end of the OTW catheter 130. Proximal movement of control wire 142 relative to the catheter shaft 132 causes the deflectable tip 144 to curve.

For coronary type applications, the deflectable tip portion 144 of the catheter 130 is about 1 to about 10 mm in length, and preferably is about 2 to about 3 mm in length. The diameter of the deflectable tip portion 144 is relatively small, from about 0.020 inches to about 0.050 inches, and is preferably about 0.030 inches to about 0.040 inches. A suitable liner 136 has a wall thickness from 0.0001 inches to about 0.005 inches, and is preferably about 0.0002 inches to about 0.0015 inches thick. The inner diameter of the liner 136 is slightly larger than the diameter of the guide wire 114, e.g., about 0.001 inches to about 0.005 inches larger. Articulation structure 160 has a length sufficient to establish a curve at the end of the catheter, and for coronary type applications is preferably about 2 to about 5 mm in length. FIG. 9D shows the junction between shaft 132 of catheter 130 and deflectable distal tip 144, including articulation structure 160.

According to another embodiment of the invention, an alternative articulation structure 160a is shown in FIGS. 8C and 8D. Articulation structure 160a includes rings 162a connected by a spine 164a. A longitudinally extending tongue 166a connects to the distal-most ring 162a′. The other rings 162a are interrupted at the location where tongue 166a extends, so that rings 162a have an essentially U-shaped configuration. The proximal end of tongue 166a connects to the control wire 142a, which then extends proximally to the proximal end of the catheter 130. Articulation structure 160a thereby integrates the control wire 142a into articulation structure 160a to, among other things, minimize profile at the distal tip 144 of the catheter 130.

FIG. 8E shows a further alternative articulation structure 160b, according to an embodiment of the invention. Structure 160b is a coil including a series of turns 162b. The control wire 142b connects to the distal-most turn 162b′ of the coil, causing the coil to curve when foreshortened. Various other articulation structures may be incorporated into any of the catheter embodiments described herein.

In use, when control wire 142 is withdrawn proximally relative to the catheter shaft 132, the articulation structure 160, 160a, 160b of distal tip 144 is deflected. Preferably, the amount of deflection is proportional to the amount of relative movement between control wire 142 and the catheter shaft 132. To facilitate control of the rotational orientation of tip 144 within the blood vessel, the catheter 130 may be rotated, or torqued, to a desired orientation.

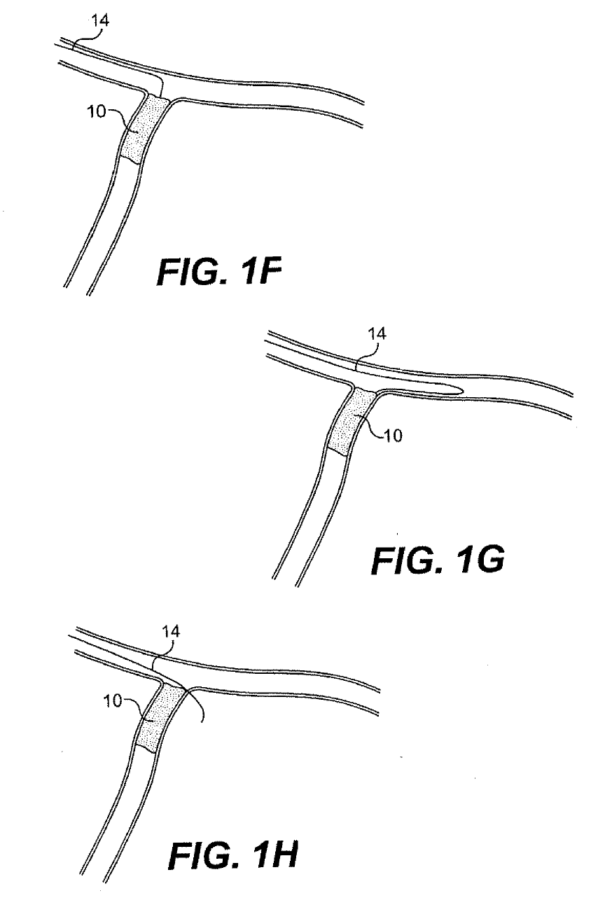

A method of use of the OTW style control catheter 130 will now be described FIGS. 2A-2C show an occluded vessel 12, and a guide wire 114 crossing an occlusion 10 through use of a control catheter 130. In this embodiment, after bare guide wire 114 unsuccessfully crosses occlusion 10 or prior to an attempt to cross occlusion 10, guide wire 114 is positioned just proximal occlusion 10, as shown in FIG. 2A. Guide wire 114 then may be extended with conventional extension wires to make it an exchange length, typically about 300 cm. Wire control catheter 130 then is loaded over the proximal end of guide wire 114 and advanced until the distal tip 144 of catheter 130 is near occlusion 10, as shown in FIG. 2B. Alternatively, a standard length (approximately 175 cm) guide wire may be pre-loaded in the guide wire (lumen 134) of catheter 130 before attempting to cross the occlusion 10. Tip 144 then is deflected into a curve or angle via control wire 142 and articulation structure 160, 160a, 160b until the distal tip 144 of catheter 130 and the guide wire 114 are parallel to the axis of occlusion 10, as shown in FIG. 2C. Fluoroscopy may be used to visualize the guide wire 114 and catheter 130 during this step if catheter tip 144 and the distal region of guide wire 114 are made of radiopaque material.

Preferably, deflectable tip 144 of wire control catheter 130 is positioned to abut occlusion 10 to provide maximum support to the flexible tip of guide wire 114, as shown in FIG. 2C. In certain cases, such as when distal tip 144 touches the side wall of vessel 12, it may be desirable to withdraw wire control catheter 130 to a proximal position, allowing guide wire 114 to be both parallel to the occlusion axis, and relatively centered with respect to occlusion 10. This is shown in FIG. 2E. Once this desired approach position of catheter 130 is achieved, wire 114 is advanced across occlusion 10 until it is in the distal vessel 12′, as shown in FIGS. 2C and 2E. If the occlusion 10 is relatively straight or relatively short, the guide wire 114 may be advanced in a single pass, as shown in FIG. 2C. However, if the occlusion 10 is curved, the guide wire 114 may be advanced incrementally, and followed by advancement of the control catheter 130. The control catheter 130 may then be used to redirect the guide wire 114 for subsequent incremental advancement. In this manner, the path that the guide wire 114 takes through the occlusion 10 may be curved to more closely follow the curvature of the occlusion 10.

If so desired, the distal tip 144 of catheter 130 may also be advanced across the lesion 10, as shown in FIG. 2D. By crossing the occlusion 10 with catheter 130, guide wire 114 can be easily exchanged for a guide wire having different characteristics, if desired. Also, contrast media may be delivered through lumen 134 to aid in confirming successful crossing of occlusion 10.

Once occlusion 10 is successfully crossed by guide wire 114, (and confirmed as described below), wire control catheter 130 is removed from guide wire 114. Conventional balloon angioplasty techniques, or any other desired treatment including placement of a stent, may then be performed to dilate occlusion 10.

FIG. 2F illustrates use of wire control catheter 130 in crossing an occlusion 10 near a bifurcation, a common and especially challenging anatomical feature for conventional crossing techniques with a guide wire. Substantially the same steps as discussed with respect to FIGS. 2A-2E may be used to advance wire 114 across occlusion 10 in FIG. 2F.

Prior to performing angioplasty or other desired treatment at occlusion 10, and the earlier removal step of the control catheter 130, the position of the distal tip of guide wire 114 should be confirmed to be in the vessel lumen 12′ distal to occlusion 10, as opposed to an external position following an inadvertent perforation or movement of guide wire 114 into the subintimal wall. If guide wire 114 has taken a path within the vessel wall, or completely external the vessel, there is a risk of cardiac tamponade. This risk is relatively low when only guide wire 114 has perforated. However, if angioplasty is performed, the perforation itself is dilated, resulting in a large leak path for arterial blood. Therefore, the practitioner should confirm that guide wire 114 has actually crossed occlusion 10 and entered the distal vessel 12′ prior to performing angioplasty or other surgical procedure. Confirmation may be done by manipulating guide wire 114 by torquing and/or axial movement, observed during fluoroscopy. Free manipulation of the tip of guide wire 114 indicates that guide wire 114 is in the distal vessel 12′. Angiography using one or more views can also indicate whether the guide wire tip is in the distal vessel 12′.

If guide wire 114 has a “j” tip on its end, the tip position may be confirmed by rotation of guide wire 114. If the tip is in the lumen 12′ distal of occlusion 10, the tip will easily rotate. However, if the tip does not freely rotate, it is likely outside the true lumen 12′. In this case, guide wire 114 can be withdrawn from occlusion 10, usually without consequence. Subsequent attempts at crossing occlusion 10 are then performed, possibly with reorientation of wire control catheter 130.

When crossing occlusion 10 with a straight-tipped guide wire 114, which more naturally tends to traverse a straight path across occlusion 10, it may be more difficult to confirm the distal tip position by mere wire rotation. Therefore, one may advance the wire control catheter 130 over guide wire 114 and through occlusion 10. Once catheter 130 is through, the straight-tipped wire 114 may be removed. A j-bend may be formed on that guide wire 114, or an alternate guide wire 114 with a j-bend may be used, and the j-tipped guide wire 114 is re-advanced through wire control catheter 130 and into the distal vessel 12′. This j-tipped wire 114 then may be manipulated to determine whether it is in the true lumen 12′. Then, wire control catheter 130 is removed, and angioplasty or other desired treatment is performed. It is preferable for the distal portion of the wire control catheter 130 to be of relatively low profile, to minimize expansion of the path traversed by guide wire 114, and therefore minimize the potential for an inadvertent wire perforation resulting in cardiac tamponade.

Embodiments of a guide wire 114 suitable for the invention include floppy, atraumatic tipped wires or any similar conventional guide wires known in the art. In addition to the support wire control catheter 130 may provide to guide wire 114, as described above, guide wires with stiffer tips may be used for additional support. In this case, after catheter 130 is positioned over the initial wire used to reach occlusion 10, that initial wire would be removed, keeping catheter 130 in position. A second guide wire with a stiffer tip then would be advanced through catheter 130, and attempts made to cross occlusion 10 with that stiffer-tipped wire.

If a stiff-tipped guide wire 114 is used to cross the lesion 10, it may be desirable to exchange that guide wire for a more flexible guide wire to finish the angioplasty procedure. Guide wires 114 are usually advanced to a position substantially distal of the lesion 10 before an angioplasty catheter is used. Therefore, physicians prefer to use a floppy tipped guide wire 114 to track down the length of the vessel 12, minimizing the chance of traumatizing or piercing the vessel 12. In that case, the wire control catheter 130 is advanced through the lesion 10, following the existing stiff-tipped guide wire 114. Once the catheter 130 crosses the lesion, the existing wire 114 is removed, and a floppy tipped wire 114 is inserted through the catheter 130, to pass through the lesion 10 and move distally down the vessel 12′. This procedure allows for the floppy tipped wire 114 to follow the path initially established by the stiff-tipped wire 114. At this point, the wire control catheter 130 is then removed, and conventional angioplasty performed.

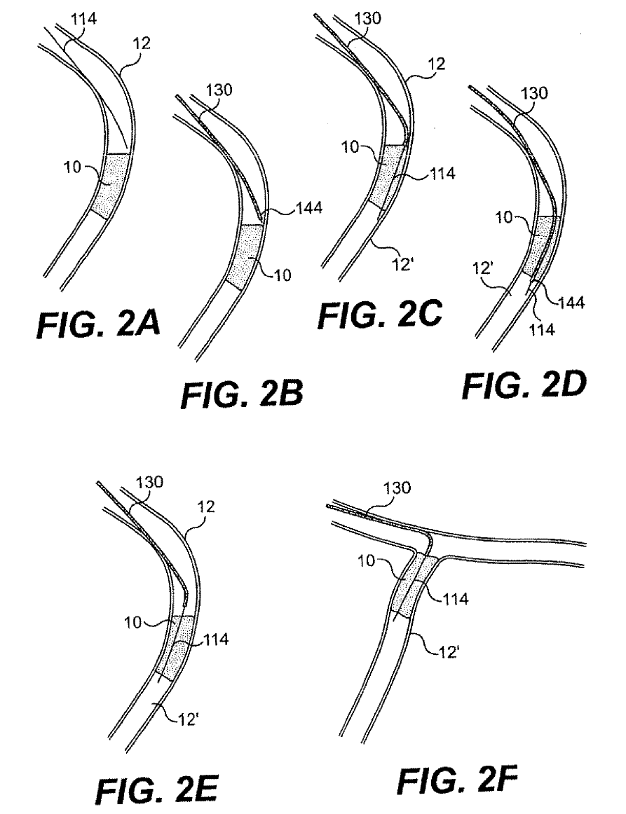

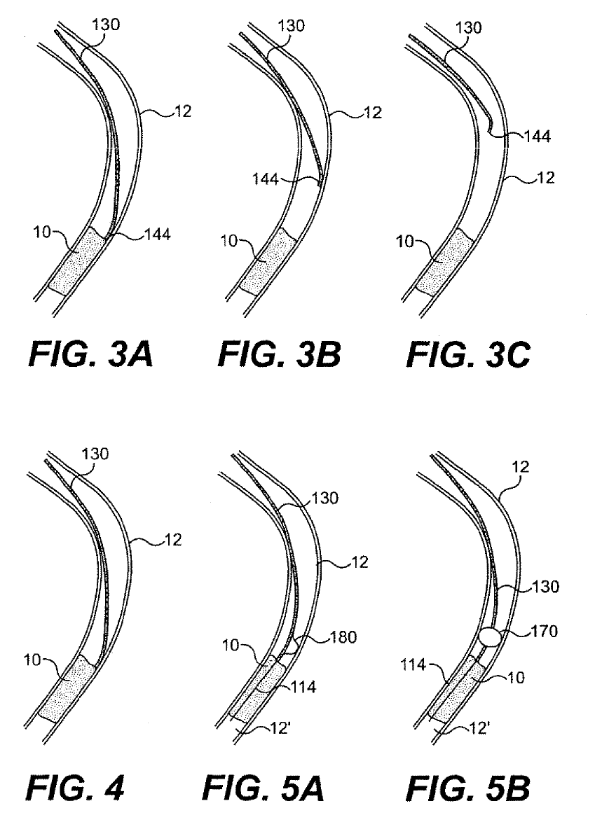

It may be desirable to position the OTW style control catheter 130 such that the guide wire 114 will have an initial alignment that is both centered and parallel to the lesion to be crossed. Proximally withdrawing the wire support catheter 130, combined with adjusting the deflection on the tip, may yield such an alignment, depending on the tortuosity of the anatomy. At the closer positions shown in FIGS. 3A, 3B and 4, the tip 144 of the wire support catheter 130 tends to contact the vessel wall 12, due to the effects of the proximal tortuosity. In some cases, depending on the degree of vessel tortuosity, the distance that the catheter 130 needs to be withdrawn may be quite large, as shown in FIG. 3C. This distance (FIG. 3C) may be too great to effectively align and support the flexible end of the guide wire 114 during the lesion crossing.

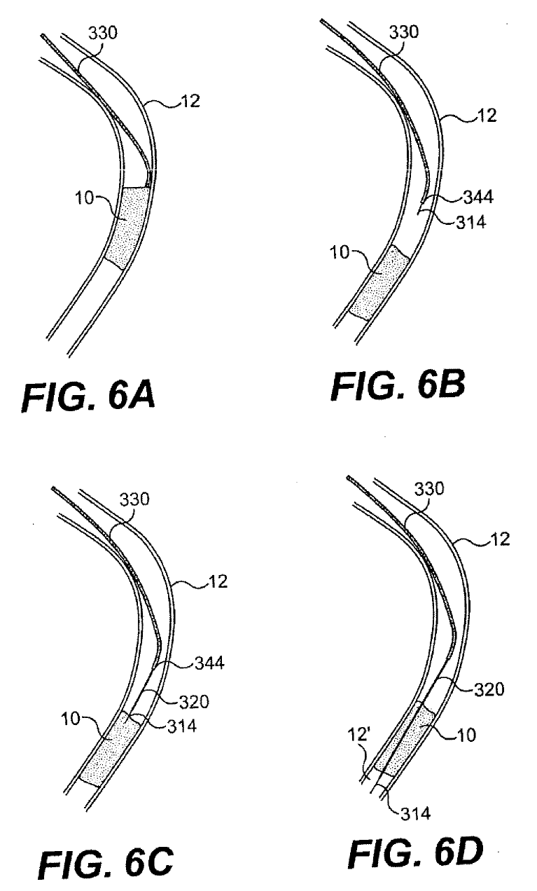

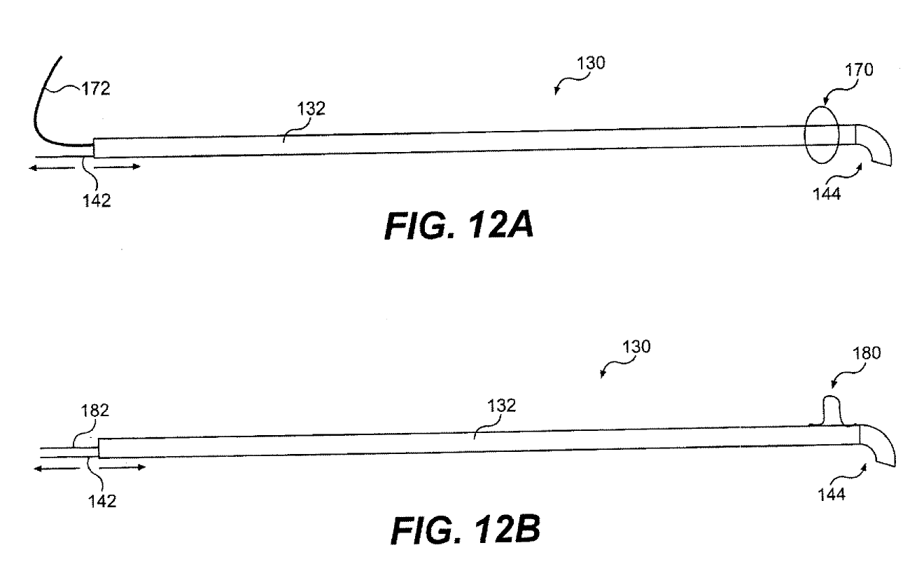

According to another aspect of the invention, the catheter 130 may include a centering element to actively position the deflectable distal tip 144 of the wire support catheter 130 towards the center of the proximal end of the lesion 10, and away from the vessel wall 12, while allowing the tip 144 to be close to the occlusion 10. As embodied herein and shown in FIG. 12A, the centering element may be an inflatable balloon 170 near the deflectable distal tip 144 of the wire control catheter 130. An inflation tube 172, defining an inflation lumen, extends within the catheter shaft 132 to the proximal end of the catheter 130. An inflation device (not shown) is utilized to inflate the balloon 170. In use, wire control catheter 130 is positioned near the occlusion 10, as shown in FIG. 2B. The balloon 170 is inflated, bringing the deflectable tip 144 of the catheter 130 towards the center of the vessel 12. The deflectable tip 144 is then articulated to align the guide wire 114 parallel to the occlusion 10, as shown in FIG. 5B. As an alternative, deflectable tip 144 also may be articulated prior to inflating balloon 170.

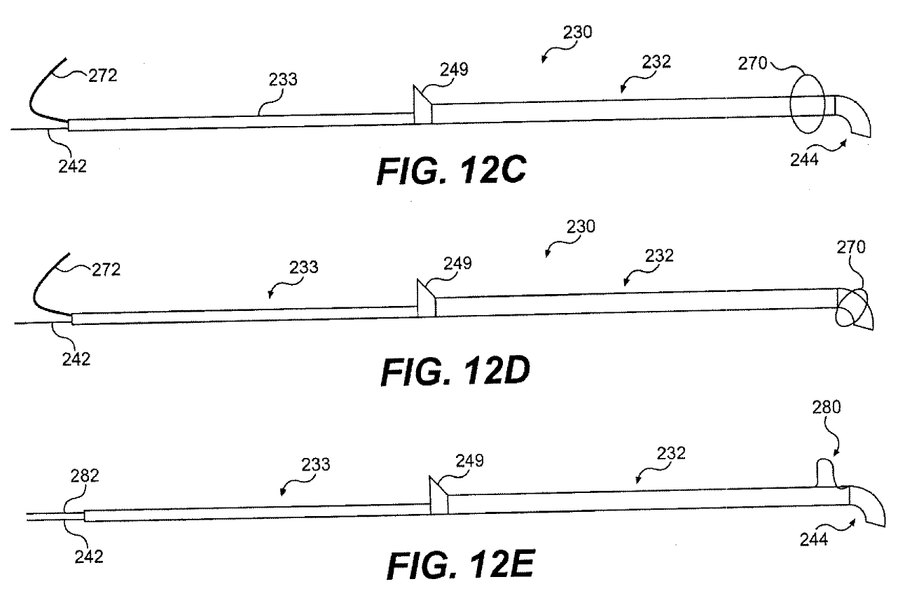

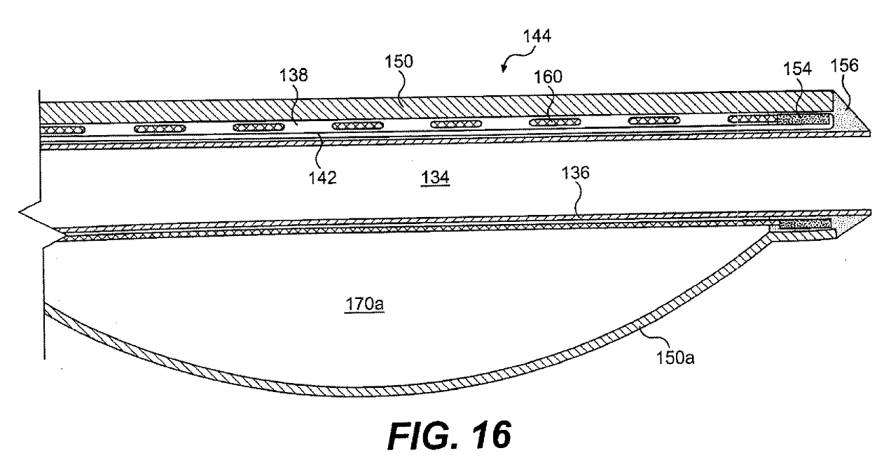

Alternatively, as shown in FIGS. 13C and 16, the inflatable balloon 170 may be positioned on the distal deflectable tip 144 of the catheter 130. FIG. 13C also shows optimal pre-dilation balloon 190, as will be described later. FIG. 16 shows a centering balloon incorporated into an articulation structure. As shown in FIG. 16 and embodied herein, the outer tube 150a is also an inflatable balloon 170a. Since the balloon 170a only needs to inflate on the side of the catheter 130 opposite the articulation curve of the directable tip, it is only necessary to provide an inflatable structure on one side of the catheter, rather than encircling the catheter 130. The tubing 150 may have a wall thickness that is thinner in an area to be inflated. In this embodiment, the control wire lumen 138 is also the inflation lumen. Upon inflation, the thinner portion of the outer tube 150a expands, causing the distal tip 144 of the wire control catheter 130 to move away from the vessel wall 12. Preferred materials for the outer tube 150a include silicone and polyurethane. To further force the balloon expansion to occur opposite the articulation curve, the balloon wall 150a can be discreetly heat bonded to the rings of the articulation structure (not shown).

According to another aspect of the invention, as shown in FIG. 12B, the centering element may include a protrusion wire 182 that emerges from the side of the wire support catheter 130 near the distal end. Preferably, the protrusion wire 182 emerges from the side of the catheter 130 opposite a direction of deflection of the deflectable tip 144, as shown in FIGS. 12B and 5A. A lumen (not shown) extends proximally from the protrusion region along the length of the catheter shaft 132. The protrusion wire 182 extends within this lumen to the proximal end of the catheter 130. An opening (not shown) is provided in a distal portion of the catheter 130, through the protrusion wire lumen, for a bent centering portion 180 of the protrusion wire 182 to extend outside of the catheter 130 and into the vessel 12. The amount that the bent centering portion 180 of protrusion wire 182 extends or protrudes into the vessel 12 is controlled by relative movement between the protrusion wire 182 and the catheter shaft 130 at the proximal end of the catheter 130.

Additionally, as shown in FIGS. 13A-13D, the catheter 130 may include a pre-dilation balloon 190. The balloon 190 is shown in an inflated state in FIG. 13A. The balloon 190 preferably has an inflated diameter of about 1.5 mm or larger, and a length of about 20 mm. The balloon 190 is preferably positioned about 2-5 cm proximal of the deflectable tip 144 of the catheter 130. This allows the guide wire 114 and catheter tip 144 to cross the lesion 10 and allows the position of the guide wire 114 and tip 144 to be verified prior to advancing the pre-dilation balloon 190 into the lesion 10. The balloon 190 is then advanced across the occlusion 10 to pre-dilate the lesion 10, which facilitates subsequent stent implantation.

FIG. 13B shows a cross-section of the proximal shaft of a catheter having the pre-dilation balloon, illustrating the additional lumen 192 used for inflation and deflation of the balloon 190. FIG. 13C shows an alternative embodiment of the catheter 130 with pre-dilation balloon 190, which also incorporates a centering balloon 170 at or near the tip 144 of the wire control catheter 130. The pre-dilation balloon 190 is shown schematically in a deflated and folded condition in FIG. 13C, as it would be when it is advanced across the occlusion 10 and before it is inflated to pre-dilate the stenosis 10. FIG. 13D shows the proximal shaft of a catheter having the pre-dilation balloon 190 and centering balloon 170, illustrating the tube 192 used for inflation and deflation of the pre-dilation balloon 190 and the tube 172 used for inflation and deflation of the centering balloon 170. In this embodiment, the centering balloon 170 could be inflated via the control wire lumen 138, or could incorporate an inflation tube 172, as shown.

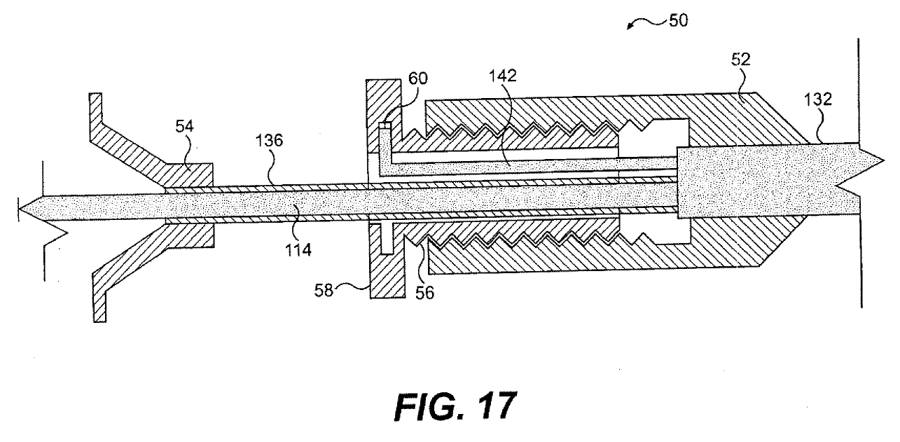

As embodied herein and shown in FIG. 17, the wire control catheter 130 connects to a handle structure 50 attached to the proximal end of the catheter 130. A base portion 52 of the handle structure 50 is connected to the proximal end of the shaft 132. The guide wire liner 136 extends proximally and has a conventional luer fitting 54, to facilitate both wire exchanging as well as contrast delivery through the guide wire lumen 134. The ability to inject contrast may be useful to assess whether the device has accessed the true lumen, as depicted in FIG. 2D. A rotating advancer 58 engages the base portion 52 of the handle structure via threads 56. The proximal end of the control wire 142 engages a channel 60 in the rotating advancer 58. Rotation of the advancer 58 relative to the base portion 52 causes relative longitudinal motion between the control wire 142 and the catheter shaft 132.

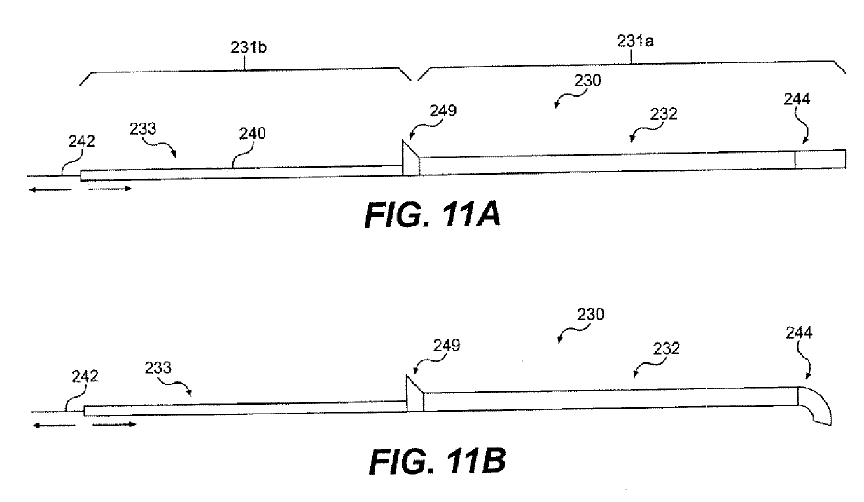

According to another aspect of the invention, the wire control catheter may not be provided with a full length guide wire lumen. Instead, as embodied herein and shown in FIGS. 11A and 11B, a monorail style wire support catheter 230 may be provided. Monorail style catheter 230 includes a distal region 231a and a proximal region 231b. Distal region 231a includes a shaft 232 similar to the shaft for the OTW style catheter 130. Shaft 232 defines a guide wire lumen 234 (FIG. 9E). The guide wire lumen 234 ends at a point significantly distal of the proximal end of catheter 230. The proximal region 231b of catheter 230 incorporates a shaft 233 having a lumen 238 (FIG. 9E) through which a control wire 242 for controlling articulation of a deflectable distal tip section 244 extends. Deflectable distal tip 244 has substantially the same structure as previously described with respect to deflectable tip 144 of catheter 130 as shown in FIG. 7. Deflectable distal tip 244 utilizes the same or similar articulation structures as those previously described with respect to FIGS. 8A-8E.

A funnel 249 may be provided at the proximal end of shaft 232 to facilitate guiding a tip of the guide wire 214 into the guide wire lumen 234, especially during guide wire exchange. Funnel 249 may be radiopaque to allow for fluoroscopic visualization of the guide wire into funnel 249. In use, a guide wire 214 extends side-by-side with the proximal region 231b of catheter 230. This type of catheter structure allows for the catheter to be advanced over the indwelling guide wire without the need to extend the guide wire to “exchange length.”

As embodied herein and shown in FIG. 9B, the shaft 232 of the catheter 230 includes a liner 236 that extends longitudinally to form the guide wire lumen 234. Surrounding the liner 236 is a wire braid structure 246, to provide torsional rigidity. The wire braid 246 is preferably metallic, made for example of a metallic ribbon of stainless steel. Preferably, the metallic material is a ribbon having the dimensions of about 0.001 inch by 0.003 to 0.008 inch. The pick count can be varied along the length of the shaft to further alter the stiffness and torsional stiffness qualities.

A tube 240 defines the control wire lumen 238 and is preferably positioned external to the braid structure 246. This structure is then encapsulated with a polymer such as polyurethane, nylon, Pebax, polyimide, PEEK, silicone, or other similar materials. The encapsulation 248 forms a smooth, outer surface of the catheter 230. Preferably, multiple sections of encapsulation 248 are utilized to change the flexibility of the shaft 232 from a distal end to a proximal end. For example, the distal most portion of the shaft may incorporate an encapsulation of a relatively flexible polymer such as a soft durometer polyurethane, and progress to more rigid polyurethanes or Pebax, progress to Nylon, and then to a polyimide encapsulation. Any number and composition of encapsulation materials are contemplated to tailor the shaft stiffness and torsional stiffness qualities at various positions along the length of the shaft.

The proximal shaft 233 of the monorail style control catheter 230 is preferably fabricated of a relatively stiff tube, such as a metallic hypotube of stainless steel. Such a proximal shaft structure has relatively high torsional stiffness. FIG. 9E shows the junction between the proximal shaft 233 and the mid-shaft 232 of the monorail catheter 230. A suitable connection between the proximal shaft 233 and the “mid-shaft” also includes a funnel shape 249, as shown in FIG. 11A and 11B.

According to another aspect of the invention, the catheter 230 may include a centering element to actively position the deflectable distal tip 244 of the wire support catheter 230 towards the center of the proximal end of the lesion 10, and away from the vessel wall 12. As embodied herein and shown in FIG. 12C, the centering element may be an inflatable balloon 270 near the deflectable distal tip 244 of the wire control catheter 230. Alternatively, as shown in FIG. 12D, the centering balloon 270 may be positioned on the deflectable distal tip 244 of catheter 230. Centering balloon 270 functions in substantially the same manner and has substantially the same structure as the centering balloon 170 previously discussed with regard to FIGS. 12A, 13C, and 16.

Alternatively, as shown in FIG. 12E, monorail style control catheter 230 may include a centering element in the form of a protrusion wire 282 that emerges as a protrusion 280 from the side of the wire support catheter 230 near the distal end. Centering protrusion wire 282 functions in substantially the same manner and has substantially the same structure as the centering protrusion wire 182 previously discussed with regard to FIG. 12B. Additionally, catheter 230 may include a pre-dilation balloon, similar to that previously described with respect to FIGS. 13A-13D.

In a typical use of monorail-style wire support catheter 230, catheter 230 is loaded onto the proximal end of the indwelling guide wire 214, either after efforts to cross the occlusion 10 with this guide wire 214 have failed or prior to an attempt to cross the occlusion 10. Wire control catheter 230 then is loaded over the proximal end of guide wire 214 and advanced until the distal tip 244 of catheter 230 is near occlusion 10. Tip 244 then is deflected into a curve or angle by pulling control wire 242 proximally relative to the catheter shaft 230, as with OTW catheter 130 described above, until the distal tip 244 of catheter 230 and the guide wire 214 are parallel to the axis of occlusion 10. Fluoroscopy may be used to visualize the guide wire 214 and catheter 230 during this step if catheter tip 244 and the distal region of guide wire 214 are made of radiopaque material. The indwelling guide wire 214, or another type of guide wire replacing the indwelling guide wire 214, is advanced to the distal end of the wire control catheter 230 and through the occlusion 10. Once the occlusion 10 is successfully crossed, the wire control catheter 230 is removed proximally off the guide wire 214. Again, since the guide wire lumen 234 of the catheter 230 is relatively short in the monorail catheter 230, the guide wire 214 may be left at its standard length. As with the OTW style wire support catheter 130, conventional angioplasty techniques, or any other desired surgical procedure, then may be performed to dilate or otherwise treat the occlusion 10.

According to another aspect of the invention, a sliding sheath catheter may be provided in combination with a control catheter. The control catheter may comprise either a monorail style catheter, such as that described in connection with FIGS. 11A and 11B, or an OTW style catheter, such as that described in connection with FIG. 10, and may further include centering elements (e.g., balloon) and/or a pre-dilation balloon as described earlier. For purposes of describing this embodiment, a monorail style catheter will be referred to, however, it should be understood that either type of control catheter may be used with this embodiment.

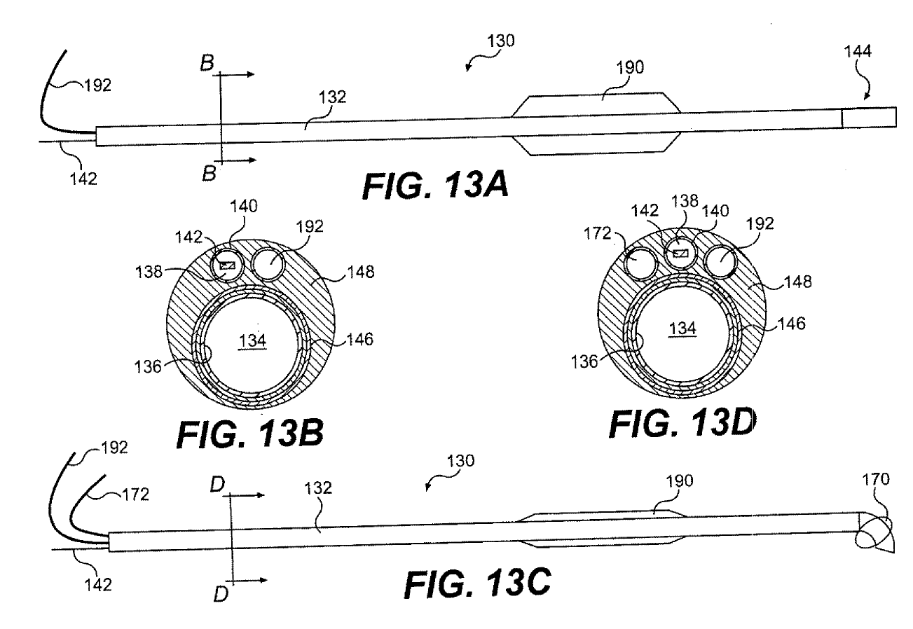

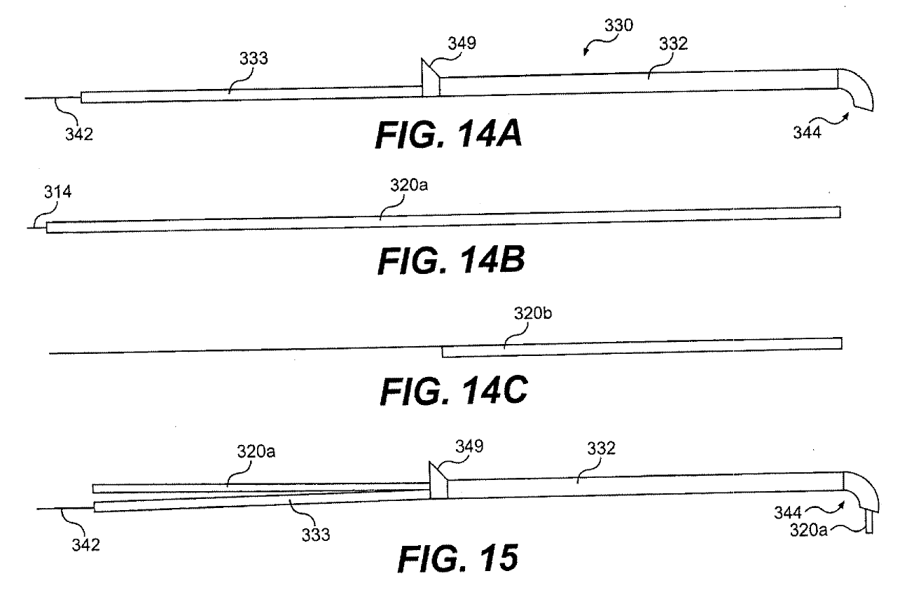

As embodied herein and shown in FIGS. 14A-15, a combination system for crossing an occlusion while minimizing dilation of the occlusion is provided. As shown in FIG. 14A, a monorail catheter 330 is provided. Also provided is a small diameter, thin advanceable sheath catheter 320. FIG. 14B illustrates a sliding sheath catheter 320a with a “full length” sheath. FIG. 14C illustrates a sliding sheath catheter 320b with a “monorail” style sheath, wherein only the distal portion of the catheter 320b incorporates a guide wire lumen. The sheath catheter 320 is sized to fit within the guide wire lumen 334 of the wire control catheter 330, and is annularly disposed between the guide wire 314 and the wire control catheter 330 as shown in FIG. 15. In a preferred embodiment, the sheath catheter 320 may be made of PTFE, HDPE, or PEEK. Other materials having similar characteristics may be used. Preferably, the sheath catheter 320 has an inner diameter of between about 0.015 inches and about 0.017 inches, and the sheath catheter 320 may have a wall thickness of approximately 0.001 inches to approximately 0.005 inches. In this embodiment, the inner diameter of the guide wire lumen 334 of the wire control catheter 330 should be larger than that for the embodiments described above.

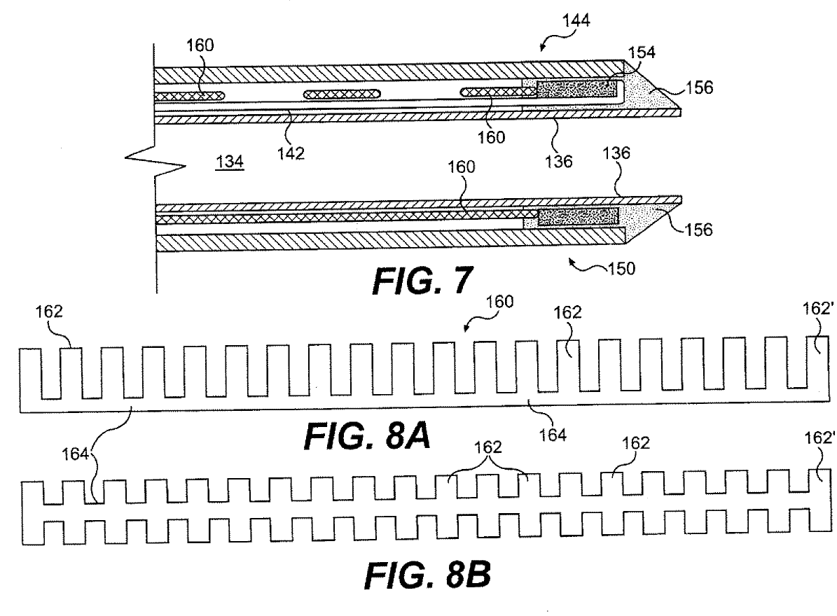

This combination system, as shown in FIG. 15, is used as described below. First, the guide wire 314 and wire control catheter 330 are positioned adjacent the lesion 10 as shown in FIG. 6A. Alternatively, to facilitate a more centered approach, the guide wire 314 and wire control catheter 330 may be positioned as illustrated in FIG. 6B. The sliding sheath 320 may also be “pre-loaded” with its distal tip near the distal tip 344 of the wire control catheter 330, or it may be subsequently loaded onto the guide wire 314 and into the wire control catheter 330 to that position. The remainder of the procedure will be described relative to the position shown in FIG. 6B. Once positioned as shown in FIG. 6B, the guide wire 314 and the sliding sheath catheter 320 are advanced to the lesion 10, as shown in FIG. 6C. Next, the guide wire 314 is advanced across the occlusion 10, being supported and guided by the sliding sheath catheter 320. The sheath catheter 320 may be advanced together with the guide wire 314 or may be advanced after the guide wire 314 is advanced through the lesion 10, to the resultant position shown in FIG. 6D. At this point, the guide wire 314 can be removed. Alternatively, it may be replaced, if necessary, for deeper advancement into the coronary tree.

The sliding sheath embodiments 320a, 320b of the invention allow crossing the total occlusion with a very small diameter, thin walled catheter 320, thus minimizing dilation of the lesion 10 beyond that done by the guide wire 314 itself. Therefore, if the path across the lesion 10 is subintimal or extravascular, little blood leakage will occur prior to confirmation of such a pathway.

While preferred embodiments of the various components of wire control catheters described include metals, such as stainless steel and platinum alloys, it is also contemplated that most or all components of wire control catheters described here could be fabricated from non-metallic components This may be important when Magnetic Resonance Imaging (MRI) is employed, during which use of these catheters is also contemplated. For example, articulation structures could be fabricated from high strength polymers, such as PEEK or polyimide. Control wires could be fabricated from the same materials, as well as high strength fibers or fiber bundles, such as nylon, polyester, ultra-high molecular weight polyethylene, Kevlar, and vectran.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1 A method for crossing a lesion in a blood vessel comprising:
providing a catheter comprising an elongate tubular member having a proximal and a distal end, a guide wire lumen extending from a proximal region and communicating with a port at the distal end, a deflectable tip portion at the distal end of the elongate tubular member comprising a plurality of spaced-apart loops arranged along a longitudinal axis, a control wire lumen extending from the proximal region to the distal end, and a control wire extending through the control wire lumen;
advancing the catheter to a region of interest;
operating the control wire to align the deflectable tip portion of the catheter with the lesion; and
advancing a guide wire through the guide wire lumen of the catheter and across the lesion.

2. The method of claim 1, wherein aligning the deflectable tip portion of the catheter comprises moving the control wire relative to the elongate tubular member of the catheter.

3. The method of claim 2, wherein the plurality of loops is connected to the control wire extending through the control wire lumen.

4. The method of claim 1, wherein aligning the deflectable tip portion further comprises deflecting the deflectable tip portion to be parallel with the axis of the lesion.

5. The method of claim 4, wherein aligning the deflectable tip portion further comprises deflecting the deflectable tip portion to be substantially centered with respect to the lesion.

6. The method of claim 1, wherein the deflectable tip portion is substantially incompressible along not more than one longitudinal axis on the circumference of the catheter,

7. The method of claim 1 wherein the deflectable tip portion is incapable of deflecting in more than one direction by operation of the control wire.

8. The method of claim 1 wherein the plurality of loops are connected along the longitudinal axis by a spine.

9. The method of claim 1, wherein the plurality of loops are a plurality of rings.

10. The method of claim 1, wherein the plurality of loops have a u-shaped configuration.

11. The method of claim 1, wherein the plurality of loops are closed loops.

12. The method of claim 1, wherein the plurality of loops are part of a coil.

13. The method of claim 1, further comprising centering the catheter prior to advancing the guide wire across the lesion.

14. The method of claim 13, wherein the catheter further comprises a centering device.

15. The method of claim 14, wherein the centering device is located near the deflectable tip portion.

16. The method of claim 14, wherein the centering device is located at the deflectable tip portion.

17. The method of claim 14, wherein the centering device is inflatable and wherein the elongate tubular member further includes an inflation lumen.

18. The method of claim 1, wherein the catheter further comprises a dilation balloon, and wherein the method further comprises expanding the balloon to dilate the lesion.

19. The method of claim 1, further comprising a funnel portion in communication with the proximal end of the guide wire lumen.

20. The method of claim 1, further comprising advancing a guide wire into the blood vessel near the region of interest in the blood vessel.

21. The method of claim 1, further comprising advancing the deflectable tip portion of the catheter across the lesion.

22. The method of claim 21, further comprising deflecting the distal tip of the wire control catheter after the catheter has crossed the lesion to determine if the guide wire is within the blood vessel lumen.

23. The method of claim 21, wherein the steps of advancing the guide wire and deflectable tip portion across the lesion comprise incrementally advancing the guide wire and the deflectable tip portion across the lesion.

24. The method of claim 1, where the region of interest is a total chronic occlusion.

25. The method of claim 1, wherein advancing the catheter to a region of interest further comprises advancing the catheter such that the deflectable tip portion abuts the lesion.

26. The method of claim 1, further comprising the steps of:
removing the catheter from the region of interest while maintaining the guide wire across the lesion;
advancing an angioplasty catheter across the lesion; and
dilating the lesion.

27. The method of claim 1, further comprising the steps of:
removing the catheter from the region of interest while maintaining the guide wire across the lesion;
advancing a stent catheter across the lesion; and
dilating the lesion with a stent.

28. The method of claim 1, wherein the region of interest is located in a coronary artery.

29. The method of claim 1, wherein the region of interest is located in a carotid artery.
